(19) United States
(12) Patent Application Publication
(10) Pub. No.: US 2009/0306201 A1
(43) Pub. Date: Dec. 10, 2009

(54) SELECTIVE INHIBITORS FOR TRANSFERASES

(75) Inventors: Danny Reinberg, Warren, NJ (US); Patrick Trojer, Burlington, MA (US); Gianluca Sbardella, Fisciano (IT)

Correspondence Address: FOX ROTHSCHILD LLP;PRINCETON PIKE CORPORATE CENTER, 2000 Market Street, Tenth Floor, Philadelphia, PA, 19103, US

(73) Assignee: University of Medicine and Dentistry of New Jersey, Somerset, NJ (US)

(21) Appl. No.: 12/306,526

(22) PCT Filed: Jun. 15, 2007
(86) PCT No.: PCT/US2007/071351

§ 371 Date: Jul. 9, 2009

Related U.S. Application Data
(60) Provisional application No. 60/815,962, filed on Jun. 23, 2006.

(51) Int. Cl. A61K 31/196; A61K 31/343; C07D 307/78; C07C 229/40; A61P 35/00

(52) U.S. Cl. 514/470; 514/567; 549/466; 562/452

(57) ABSTRACT

A pharmaceutical composition comprising a compound of formula I or II and a pharmaceutically acceptable carrier. Methods for treating a proliferative disorder mediated by a methyl transferase comprising administering an anti-proliferative effective amount of the compound of formula I or II are also presented.

### CROSS-REFERENCE TO RELATED APPLICATIONS

This application claims priority under 35 U.S.C. 119(e) to U.S. Provisional Application Ser. No. 60/815,962, which was filed on Jun. 23, 2006. The disclosure of this application is incorporated herein by reference.

### BACKGROUND OF THE INVENTION

Chromatin, the organized assemblage of nuclear DNA and histone proteins, is the basis for a multitude of vital nuclear processes including regulation of transcription, replication, DNA-damage repair and progression through the cell cycle. The basic unit of chromatin is the nucleosome, consisting of an octamer of histones H2A, H2B, H3 and H4, as well as 146 base pairs of DNA, which wraps around this histone core. Recently, a number of factors, chromatin-modifying enzymes, have been identified that play an important role in maintaining the dynamic equilibrium of chromatin.

The amino termini of histones (histone tails) are accessible, unstructured domains that protrude out of the nucleosomes. Histones, especially residues of the amino termini of histones H3 and H4 and the amino and carboxyl termini of histones H2A, H₂B and H1, are susceptible to a variety of post-translational modifications. One type of modification, lysine methylation, is catalyzed by histone lysine methyltransferases (HKMTs). Six lysine residues of histones H3 and H4 have been identified to be the main target sites of methylation: lysines 4, 9, 27, 36, 79 of histone H3 and lysine 20 of histone H4. Histone lysine methylation is considerably different from the other types of modifications. It is regarded more stable than other histone modifications despite the recent discovery of histone lysine demethylases. HKMTs have a high specificity regarding a particular methylation site. Moreover, in higher organisms HKMTs have been identified that only catalyze one degree of methylation on a given lysine residue. The fact that histone lysine methylation comes in three degrees provides the basis for a highly complex regulatory system. In contrast to other modifications, which can be either present or absent, histone lysine methylation can be absent or present in a mono-, di- or tri-methylated form. In principle this suggests for each residue a quadruple instead of a binary readout.

In every multicellular organism, cells acquire specific functions through a differentiation state determined by the cell specific pattern of gene expression, which in turn is established and maintained through the differential packaging of DNA into chromatin. HKMTs play a key role in establishing and maintaining stable gene expression patterns during cellular differentiation and embryonic development, impacting on the regulation of both transcriptional activation and repression dependent on the particular site and degree of methylation.

Importantly, histone lysine methylation and HKMTs have been implicated in disease. Studies showed global alterations of histone modifications in cancerous cells compared to the normal cellular state. For instance, histone lysine methylation patterns were found to be completely perturbed in various types of cancer. Hence, specific loss in histone H4 lysine 16 acetylation (H4K16ac) or H4 lysine 20 trimethylation (H4K20me3) have been suggested to be a common mark of human cancer [Fraga et al. 2005].

Moreover, several HKMTs have been shown to be overexpressed in cancer cells. For example EZH2 (a HKMT mediating H3K27 methylation) has been linked to invasive prostate and breast cancer; RIZ1 (mediating H3K9 methylation) has been identified as tumor suppressor and MLL1 (mediating H3K4 methylation) is implicated in specific types of myeloid leukaemia.

### SUMMARY OF THE INVENTION

The present invention relates to pharmaceutical compositions, which include (a) an effective amount of a compound of Formula I:

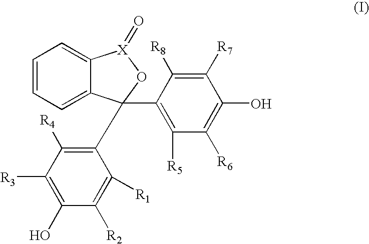

wherein

R₁-R₈ are independently selected from H, (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂Rₐ, SOₘRₐ, S(O)ₘNRₐR_{b}, P(═O)(ORₐ)(Rₐ), and Het, wherein (C₁₋₇)alkyl or (C₃₋₁₂)cycloalkyl are each independently optionally substituted with from 1 to 5 aryl, Het, ORₐ, halo, NO₂, NRₐR_{b}, cyano, CONRₐR_{b}, CO₂Rₐ, SOₘRₐ, S(O)ₘNRₐR_{b}, or P(═O)(ORₐ)(Rₐ);

X is C or S;

Rₐ and R_{b} are each independently H, (C₁₋₇)alkyl, (C₃₋₁₂)cycloalkyl, (C₂₋₇)alkanoyl, (C₂₋₇)alkanoyloxy, or aryl, or Rₐ and R_{b} together with a nitrogen to which they are attached form a Het;

m is 0, 1, or 2;

n is 0, 1, 2, 3, or 4;

or a derivative of the compound selected from N-oxide derivatives, prodrug derivatives, protected derivatives, isomers, and mixtures of isomers of the compound; or a pharmaceutically acceptable salt or solvate of the compound or the derivative; and (b) a pharmaceutically acceptable carrier.

Also presented are pharmaceutical compositions, which include (a) an effective amount of a compound of Formula II:

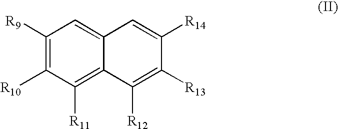

wherein

R₉-R₁₄ are independently selected from H, (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂Rₐ, SOₘRₐ, S(O)ₘNRₐR_{b}, P(═O)(ORₐ)(Rₐ), —N═N-aryl, NHC(═O)Rₐ, Het, and R₁₁ and R₁₂ together are —OC(═O)—NH—, wherein (C₁₋₇)alkyl or (C₃₋₁₂)cycloalkyl are each independently optionally substituted with from 1 to 5 aryl, Het, ORₐ, halo, NO₂, NRₐR_{b}; cyano, CONRₐR_{b}, CO₂Rₐ, SOₘRₐ, S(O)ₘNRₐR_{b}, or P(═O)(ORₐ)(Rₐ);

Rₐ and R_{b} are each independently H, (C₁₋₇)alkyl, (C₃₋₁₂)cycloalkyl, (C₂₋₇)alkanoyl, (C₂₋₇)alkanoyloxy, aryl, or Na;

m is 0, 1, 2, or 3;

n is 0, 1, 2, 3, or 4; or

a derivative of the compound selected from N-oxide derivatives, prodrug derivatives, protected derivatives, isomers, and mixtures of isomers of the compound; or a pharmaceutically acceptable salt or solvate of the compound or the derivative; and (b) a pharmaceutically acceptable carrier.

Also presented are methods for treating a proliferative disorder mediated by a methyl transferase by administering an anti-proliferative effective amount of a compound of formula I or II to a patient in need thereof.

The present invention also relates to compounds of formula III:

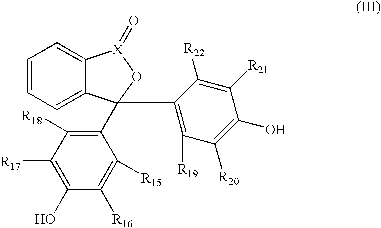

wherein

R₁₅-R₂₂ are independently selected from H, (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂Rₐ, SOₘRₐ, S(O)ₘNRₐR_{b}, P(═O)(ORₐ)(Rₐ), and Het, wherein (C₁₋₇)alkyl or (C₃₋₁₂)cycloalkyl are each independently optionally substituted with from 1 to 5 aryl, Het, ORₐ, halo, NO₂, NRₐR_{b}, cyano, CONRₐR_{b}, CO₂Rₐ, SOₘRₐ, S(O)ₘNRₐR_{b}, or P(═O)(ORₐ)(Rₐ);

X is C or S;

Rₐ and R_{b} are each independently H, (C₁₋₇)alkyl, (C₃₋₁₂)cycloalkyl, (C₂₋₇)alkanoyl, (C₂₋₇)alkanoyloxy, or aryl, or Rₐ and R_{b} together with a nitrogen to which they are attached form a Het;

m is 0, 1, or 2;

n is 0, 1, 2, 3, or 4;

or a derivative of the compound selected from N-oxide derivatives, prodrug derivatives, protected derivatives, isomers, and mixtures of isomers of the compound; or a pharmaceutically acceptable salt or solvate of the compound or the derivative;

provided that at least one of R₁₅, R₁₈, R₁₉, and R₂₂ is halo.

Also presented are compounds of formula IV:

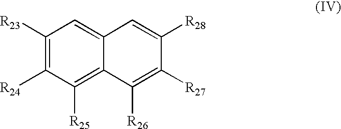

wherein

R₂₁-R₂₈ are independently selected from H, (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂Rₐ, SOₘRₐ, S(O)ₘNRₐR_{b}, P(═O)(ORₐ)(Rₐ), —N═N-aryl, NHC(═O)Rₐ, Het, and R₁₁ and R₁₂ together are —OC(═O)—NH—, wherein (C₁₋₇)alkyl or (C₃₋₁₂)cycloalkyl are each independently optionally substituted with from 1 to 5 aryl, Het, ORₐ, halo, NO₂, NRₐR_{b}, cyano, CONRₐR_{b}, CO₂Rₐ, SOₘRₐ, S(O)ₘNRₐR_{b}, or P(═O)(ORₐ)(Rₐ);

Rₐ and R_{b} are each independently H, (C₁₋₇)alkyl, (C₃₋₁₂)cycloalkyl, (C₂₋₇)alkanoyl, (C₂₋₇)alkanoyloxy, aryl, or Na;

m is 0, 1, 2, or 3;

n is 0, 1, 2, 3, or 4; or

a derivative of said compound selected from N-oxide derivatives, prodrug derivatives, protected derivatives, isomers, and mixtures of isomers of the compound; or a pharmaceutical acceptable salt or solvate of the compound or the derivative;

provided that when: (a) at least one of R₂₄ and R₂₇ is H; (b) at least one of R₂₃ and R₂₈ is H, CO₂H, OC(═O)(C₁₋₇)alkyl, or OC(═O)Na, or both R₂₃ and R₂₈ are SO₃Rₐ, and (b) R₂₅ is H, OH, or OC(═O)(C₁₋₇)alkyl and R₂₆ is H, OH, OC(═O)(C₁₋₇)alkyl, or N═N—Ar, wherein Ar is:

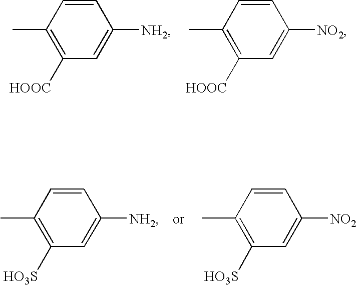

### or (c) R₂₅ is NH₂ and Y is H,

then R₂₃ and R₂₈ are independently selected from (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂R_{c}, SOₚR_{c}, S(O)ₘNRₐR_{b}, P(═O)(ORₐ)(Rₐ), —N═N-aryl, NHC(═O)Rₐ, Het, and R₂₅ and R₂₆ together are —OC(═O)—NH—.

wherein

(C₁₋₇)alkyl or (C₃₋₁₂)cycloalkyl are each independently optionally substituted with from 1 to 5 aryl, Het, ORₐ, halo, NO₂, NRₐR_{b}, cyano, CONRₐR_{b}, CO₂Rₐ, SOₘRₐ, S(O)ₘNRₐR_{b}, or P(═O)(ORₐ)(Rₐ),

R_{c} is (C₃₋₁₂)cycloalkyl, (C₂₋₇)alkanoyl, (C₂₋₇)alkanoyloxy, or aryl, and

p is 0, 1, or 2.

### BRIEF DESCRIPTION OF THE DRAWINGS

FIG. 1 provides the results of inhibitor assays with thymolphthalein (EPI-0009) in vitro;

FIG. 2 provides the results of inhibitor assays with H-acid (EPI-0023) in vitro;

FIG. 3 provides the results of toxicity tests of thymolphthalein (EPI-0009) and H-acid (EPI-0023) in HeLa cells; and

FIG. 4 illustrates the growth curve for HeLa cells treated with DMSO or thymolphthalein (EPI-0009).

### DETAILED DESCRIPTION OF THE INVENTION

The present invention relates to compositions for inhibiting a methyltransferase in a patient. Also presented are methods for treating a proliferative disorder mediated by a methyl transferase in a patient.

As used above, and throughout the description of the invention, the following terms, unless otherwise indicated, shall be understood to have the following meanings:

“Patient” means a mammal including a human.

“Effective amount” means an amount of compound of the present invention effective for inhibiting a methyltransferase, and thus producing the desired therapeutic effect.

“Treat” or “treatment” or “treating” mean to lessen, eliminate, inhibit, improve, alter, or prevent a disease or condition, for example by administration of compound of the present invention.

“Alkyl” means aliphatic hydrocarbon group which may be branched or straight-chained having about 1 to about 10 carbon atoms. Preferred alkyl is “lower alkyl” having about 1 to about 3 carbon atoms; more preferred is methyl. Branched means that one or more lower alkyl groups such as methyl, ethyl, or propyl are attached to a linear alkyl chain. The alkyl group is also optionally substituted by alkoxy, halo, carboxy, hydroxy or RₑR_{f}N— (wherein Rₑ and R_{f} are independently hydrogen or alkyl, or Rₑ and R_{f} taken together with the nitrogen atom to which Rₑ and R_{f} are attached form azaheterocyclyl); and preferably optionally substituted by fluoro. Examples of alkyl include methyl, fluoromethyl, difluoromethyl, trifluoromethyl, ethyl, n-propyl, isopropyl, butyl, sec-butyl, t-butyl, amyl and hexyl.

“Cycloalkyl” means a non-aromatic monocyclic ring system of about 3 to about 7 carbon atoms. Preferred monocyclic cycloalkyl rings include cyclopentyl, cyclohexyl, and cycloheptyl; more preferred are cyclohexyl and cyclopentyl.

“Aryl” means aromatic carbocyclic radical containing about 6 to about 10 carbon atoms. Exemplary aryl include phenyl or naphthyl, or phenyl or naphthyl substituted with one or more aryl group substituents which may be the same or different, where “aryl group substituent” includes hydrogen, hydroxy, halo, alkyl, alkoxy, carboxy, alkoxycarbonyl or Y₁Y₂NCO—, wherein Y₁ and Y₂ are independently hydrogen or alkyl.

“Het” is a four- (4), five- (5), six- (6), or seven- (7) membered saturated or unsaturated heterocyclic ring having 1, 2, 3, or 4 heteroatoms selected from the group consisting of oxy, thio, sulfinyl, sulfonyl, and nitrogen, which ring is optionally fused to a benzene ring. Het includes “heteroaryl,” which encompasses about a 5- to about a 10-membered aromatic monocyclic or bicyclic hydrocarbon ring system in which one to three of the atoms in a monocyclic ring system, and one to four of the atoms in a bicyclic ring system, is/are elements(s) other than carbon, for example nitrogen, oxygen or sulfur. The “heteroaryl” may also be substituted by one or more of the above-mentioned “aryl group substituents”. Exemplary heteroaryl groups include substituted pyrazinyl, furanyl, thienyl, pyridyl, pyrimidinyl, isoxazblyl, isothiazolyl, oxazolyl, thiazoly, pyrazolyl, furazanyl, pyrrolyl, imidazo[2,1-b]thiazolyl, benzofurzanyl, indolyl, azaindolyl, benzimidazolyl, benzothienyl, quinolinyl, imidazolyl and isoquinolinyl.

“Acyl” means an H—CO— or alkyl-CO— group in which the alkyl group is as previously described. Preferred acyls contain a lower alkyl. Exemplary acyl groups include formyl, acetyl, propanoyl, 2-methylpropanoyl, butanoyl and caproyl.

“Alkoxy” means an alkyl-O— group in which the alkyl group is as previously described. Preferred alkoxy is “lower alkoxy” having about 1 to about 3 carbon atoms; more preferred is methoxy. The alkoxy may be optionally substituted by one or more alkoxy, carboxy, alkoxycarbonyl, carboxyaryl or RₑR_{f}N— (wherein Rₑ and R_{f} are as defined above). Exemplary alkoxy groups include methoxy, ethoxy, n-propoxy, i-propoxy, n-butoxy, heptoxy, 2-(morpholin-4-yl)ethoxy and 2-(ethoxy)ethoxy.

“Aryloxy” means aryl-O— group in which the aryl group is as previously described.

“Acyloxy” means and acyl-O— group in which the acyl group is as previously described.

“Carboxy” means a HO(O)C— (carboxylic acid) group.

“RₑR_{f}N—” means a substituted or unsubstituted amino group, wherein Rₑ and R_{f} are as previously described. Exemplary groups include amino(H₂N—), methylamino, ethylmethylamino, dimethylamino and diethylamino.

“RₑR_{f}NCO—” means a substituted or unsubstituted carbomoyl group, wherein Rₑ and R_{f} are as previously described. Exemplary groups are carbamoyl(H₂NCO—) are dimethylaminocarbamoyl(Me₂NCO—).

“AcylRₑN—” means an acylamino group wherein Rₑ and acyl are as defined herein.

“Halo” means fluoro, chloro, bromo, or iodo. Preferred are fluoro, chloro or bromo, and more preferred are fluoro or chloro.

“Prodrug” means a form of the compound of formula I suitable for administration to a patient without undue toxicity, irritation, allergic response, and the like, and effective for their intended use. A prodrug is transformed in vivo to yield the parent compound of the above formula, for example by hydrolysis in blood. A thorough discussion is provided in T. Higuchi and V. Stella, Pro-drugs as Novel Delivery Systems, Vol. 14 of the A. C. S. Symposium Series, and in Edward B. Roche, et., Bioreversible Carriers in Drug Design, American Pharmaceutical Association and Pergamon Press, 1987, both of which are incorporated herein by reference.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include ethanolates, methanolates, and the like. “Hydrate” is a solvate wherein the solvent molecule(s) is/are H₂O.

“Substituent of a ring structure” means any atom or group of atoms bonded to a ring in a molecule.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, tautomeric, or stereoisomeric form, or mixture thereof, of a compound of the invention, which possesses the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

One embodiment of the present invention is a pharmaceutical composition, wherein R₁ and R₉ are methyl; R₂, R₄, R₅, and R₇ are hydrogen; and R₃ and R₆ are (CH₃)₂CH. The compound according to this embodiment is also referred to herein as “thymolphthalein.”

Yet another embodiment of the present invention is a pharmaceutical composition, which includes a compound of formula II, wherein R₉ is H and R₁₄ is selected from CO₂Rₐ and SOₘRₐ. An additional embodiment is a pharmaceutical composition, wherein R₉ is selected from CO₂Rₐ and SOₘRₐ and R₁₄ is H. Yet another embodiment of the present invention is a pharmaceutical composition, wherein R₉ and R₁₄ are independently selected from CO₂Rₐ and SOₘRₐ. In an additional embodiment, R₁₀ is H and R₁₃ is —N═N-aryl. In another embodiment, R₁₁ and R₁₂ are independently selected from H, OH, SH, NH₂, CO₂Alk, NHC(═O)Alk, —N═N-aryl. In yet another embodiment, R₁₁ and R₁₂ together are —OC(═O)—NH—.

Another embodiment of the present invention is a pharmaceutical composition, wherein the compound of formula II is:

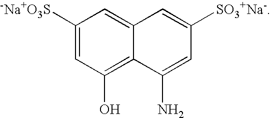

The compound according to this embodiment is also referred to herein as “H-acid.”

An additional embodiment is a pharmaceutical composition, wherein the compound of formula II is:

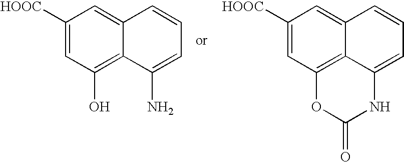

It is to be understood that this invention covers all appropriate combinations of the particular and preferred groupings referred to herein.

The present invention also includes compounds of formula III:

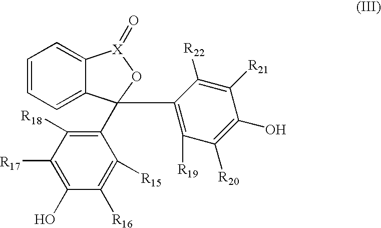

wherein

R₁₅-R₂₂ are independently selected from H, (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂Rₐ, SOₘRₐ, S(O)ₘNRₐR_{b}, P(═O)(ORₐ)(Rₐ), and Het, wherein (C₁₋₇)alkyl or (C₃₋₁₂)cycloalkyl are each independently optionally substituted with from 1 to 5 aryl, Het, ORₐ, halo, NO₂, NRₐR_{b}, cyano, CONRₐR_{b}, CO₂Rₐ, SOₘRₐ, S(O)ₘNRₐR_{b}, or P(═O)(ORₐ)(Rₐ);

X is C or S;

Rₐ and R_{b} are each independently H, (C₁₋₇)alkyl, (C₃₋₁₂)cycloalkyl, (C₂₋₇)alkanoyl, (C₂₋₇)alkanoyloxy, or aryl, or Rₐ and R_{b} together with a nitrogen to which they are attached form a Het;

m is 0, 1, or 2;

n is 0, 1, 2, 3, or 4;

or a derivative of the compound selected from N-oxide derivatives, prodrug derivatives, protected derivatives, isomers, and mixtures of isomers of the compound; or a pharmaceutically acceptable salt or solvate of the compound or the derivative;

provided that at least one of R₁₅, R₁₈, R₁₉, and R₂₂ is halo.

The present invention also includes compounds of formula IV:

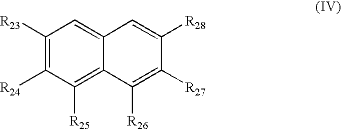

wherein

R₂₃-R₂₈ are independently selected from H, (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂Rₐ, SOₘRₐ, S(O)ₘNRₐR_{b}, P(═O)(ORₐ)(Rₐ), —N═N-aryl, NHC(═O)R_{a, Het, and R}₁₁ and R₁₂ together are —OC(═O)—NH—, wherein (C₁₋₇)alkyl or (C₃₋₁₂)cycloalkyl are each independently optionally substituted with from 1 to 5 aryl, Het, ORₐ, halo, NO₂, NRₐR_{b}, cyano, CONRₐR_{b}, CO₂Rₐ, SOₘRₐ, S(O)ₘNRₐR_{b}, or P(═O)(ORₐ)(Rₐ);

Rₐ and R_{b} are each independently H, (C₁₋₇)alkyl, (C₃₋₁₂)cycloalkyl, (C₂₋₇)alkanoyl, (C₂₋₇)alkanoyloxy, aryl, or Na;

m is 0, 1, 2, or 3;

n is 0, 1, 2, 3, or 4; or

a derivative of the compound selected from N-oxide derivatives, prodrug derivatives, protected derivatives, isomers, and mixtures of isomers of the compound; or a pharmaceutically acceptable salt or solvate of the compound or the derivative;

provided that when: (a) at least one of R₂₄ and R₂₇ is H; (b) at least one of R₂₃ and R₂₈ is H, CO₂H, OC(═O)(C₁₋₇)alkyl, or OC(═O)Na, or both R₂₃ and R₂₈ are SO₃Rₐ, and (b) R₂₅ is H, OH, or OC(═O)(C₁₋₇)alkyl and R₂₆ is H, OH, OC(═O)(C₁₋₇)alkyl, or N═N—Ar, wherein Ar is:

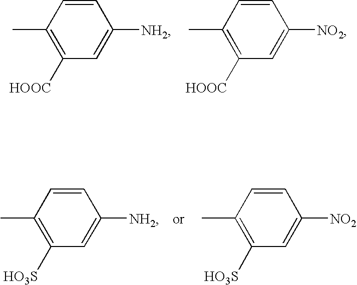

### or (c) R₂₅ is NH₂ and Y is H,

then R₂₃ and R₂₈ are independently selected from the group consisting of (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂R_{c}, SOₚR_{c}, S(O)ₘNRₐR_{b}, P(═O)(ORₐ)(Rₐ), —N═N-aryl, NHC(═O)Rₐ, Het, and R₂₅ and R₂₆ together are —OC(═O)—NH—,

wherein

(C₁₋₇)alkyl or (C₃₋₁₂)cycloalkyl are each independently optionally substituted with from 1 to 0.5 aryl, Het, ORₐ, halo, NO₂, NRₐR_{b}, cyano, CONRₐR_{b}, CO₂Rₐ, SOₘRₐ, S(O)ₘNRₐR_{b}, or P(═O)(ORₐ)(Rₐ),

R_{c} is (C₃₋₁₂)cycloalkyl, (C₂₋₇)alkanoyl, (C₂₋₇)alkanoyloxy, or aryl, and

p is 0, 1, or 2.

The compounds of this invention may be prepared by employing procedures known in the literature starting from known compounds or readily prepared intermediates. Exemplary preparation schemes are set forth in the Examples section. Alternatively, compounds, for example, thymolphthalein (Pfaltz & Bauer, Inc., Westbury, Conn.), H-acid (AK Scientific, Inc., Mountain View, Calif.), H-acid monosodium hydrate (Sigma-Aldrich, St. Louis, Mo.), H-acid monosodium salt (VWR International, Inc., West Chester, Pa.), and the like can be obtained from commercial suppliers.

The compounds of formulas I-IV are included in pharmaceutical compositions to treat, for example, a condition mediated by a methyltransferase in a patient. Examples of targeted methyltransferases include histone lysine methyltransferases (HKMTs), for example EZH2 and PRSET7. Conditions mediated by a methyltransferase include cancer, for example, prostate cancer and breast cancer.

Also provided is a method for treating a proliferative disorder mediated by a methyl transferase by administering an anti-proliferative effective amount of a pharmaceutical composition, which includes the compound of formulas I-IV, to a patient in need thereof.

In practice, a composition containing a compound of formulas I-IV may be administered in any variety of suitable forms, for example, by inhalation, topically, parenterally, rectally, or orally. More specific routes of administration include intravenous, intramuscular, subcutaneous, intraocular, intrasynovial, colonical, peritoneal, transepithelial including transdermal, ophthalmic, sublingual, buccal, dermal, ocular, nasal inhalation via insufflation, and aerosol.

A composition containing a compound of formulas I-IV may be presented in forms permitting administration by the most suitable route. The invention also relates to administering compositions containing a compound of formulas I-IV which is suitable for use as a medicament in a patient. These compositions may be prepared according to the customary methods, using one or more pharmaceutically acceptable adjuvants or excipients. The adjuvants comprise, inter alia, diluents, sterile aqueous media and the various non-toxic organic solvents. The compositions may be presented in the form of oral dosage forms, or injectable solutions, or suspensions.

The choice of vehicle and the compound of formulas I-IV in the vehicle are generally determined in accordance with the solubility and chemical properties of the product, the particular mode of administration and the provisions to be observed in pharmaceutical practice. When aqueous suspensions are used they may contain emulsifying agents or agents which facilitate suspension. Diluents such as sucrose, ethanol, polyols such as polyethylene glycol, propylene glycol and glycerol, and chloroform or mixtures thereof may also be used. In addition, the compound of formulas I-IV may be incorporated into sustained-release preparations and formulations.

For parenteral administration, emulsions, suspensions or solutions of the compounds according to the invention in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are used. The injectable forms must be fluid to the extent that it can be easily syringed, and proper fluidity can be maintained, for example, by the use of a coating such as lecithin, by the maintenance of the required particle size in the case of dispersion and by the use of surfactants. Prolonged absorption of the injectable compositions can be brought about by use of agents delaying absorption, for example, aluminum monostearate and gelatin. The solutions of the salts of the products according to the invention are especially useful for administration by intramuscular or subcutaneous injection. Solutions of the compound of formulas I-IV as a free base or pharmacologically acceptable salt can be prepared in water suitably mixed with a surfactant such as hydroxypropyl-cellulose. Dispersion can also be prepared in glycerol, liquid polyethylene glycols, and mixtures thereof and in oils. The aqueous solutions, also comprising solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation, microfiltration, and/or by various antibacterial and antifungal agents, for example, parabens, chlorobutanol, phenol, sorbic acid, thimerosal, and the like.

Sterile injectable solutions are prepared by incorporating the compound of formulas I-IV in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique, which yield a powder of the active ingredient plus any additional desired ingredient from previously sterile-filtered solution thereof.

Topical administration, gels (water or alcohol based), creams or ointments containing the compound of formulas I-IV may be used. The compound of formulas I-IV may be also incorporated in a gel or matrix base for application in a patch, which would allow a controlled release of compound through transdermal barrier.

For administration by inhalation, the compound of formulas I-IV may be dissolved or suspended in a suitable carrier for use in a nebulizer or a suspension or solution aerosol, or may be absorbed or adsorbed onto a suitable solid carrier for use in a dry powder inhaler.

The percentage of compound of formulas I-IV in the compositions used in the present invention may be varied, it being necessary that it should constitute a proportion such that a suitable dosage shall be obtained. Obviously, several unit dosage forms may be administered at about the same time. A dose employed may be determined by a physician or qualified medical professional, and depends upon the desired therapeutic effect, the route of administration and the duration of the treatment, and the condition of the patient. In the adult, the doses are generally from about 0.001 to about 50, preferably about 0.001 to about 5, mg/kg body weight per day by inhalation, from about 0.01 to about 100, preferably 0.1 to 70, more especially 0.5 to 10, mg/kg body weight per day by oral administration, and from about 0.001 to about 10, preferably 0.01 to 10, mg/kg body weight per day by intravenous administration. In each particular case, the doses are determined in accordance with the factors distinctive to the patient to be treated, such as age, weight, general state of health and other characteristics, which can influence the efficacy of the compound according to the invention.

The compound of formulas I-IV used in the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the compound of formulas I-IV may be administered 1 to 4 times per day. Of course, for other patients, it will be necessary to prescribe not more than one or two doses per day.

The following non-limiting examples set forth hereinbelow illustrate certain aspects of the invention.

### EXAMPLES

### Example 1

### Screening of Compounds Against Various Histone Lysine Methyltransferases (HKMTs) Using a Histone Methyltransferase (HMT) Assay

Compounds were initially screened for their ability to inhibit the activity of various lysine methyltransferases in vitro using a biochemical HMT-assay. Mammalian HKMTs were produced and purified as recombinant affinity-tagged proteins using a bacterial or baculovirus expression system.

The HKMT-inhibitor assay was as follows: In a final reaction volume of 25 μl, 50-100 ng recombinant HKMT protein was incubated at 30° C. for 60 minutes in a reaction buffer containing 50 mM Tris-HCl, pH 8.5, 5 mM MgCl₂, 4 mM DTT, 1 μM [³H]-labeled S-adenosyl-L-methionine ([³H]SAM, 82.0 Ci/mmol, 1.0 mCi/ml, Amersham Pharmacia Biotech) and 2 μg of substrate (histone octamers or oligonucleosomes). A constant HKMT protein concentration was tested against increasing amounts of compound dissolved in DMSO. As a control an HKMT-inhibitor assay was performed in the presence of 0.5 μl of DMSO (end concentration of DMSO in the reaction= 2%). The reaction was stopped by the addition of 6 μl 5×SDS sample buffer (60 mM Tris-HCl, pH 6.8, 25% glycerol, 2% SDS, 0.05% bromphenol blue, 14.4 mM 6-Mercaptoethanol), and the proteins were separated on a 12% SDS-polyacrylamide gel. Proteins were transferred onto an Immobilon-P membrane (Millipore) and visualized by Coomassie blue staining. The membrane was then sprayed with EN3HANCE (NEN) and exposed to Kodak XAR film overnight. Coomassie blue stained histone bands were cut from the membrane and subjected to scintillation counting. All assays were performed in duplicate and the average of two experiments was used for the calculation of IC₅₀ values. Only compounds with IC₅₀<10 μM were selected for further experiments. Results are shown in Table 1:

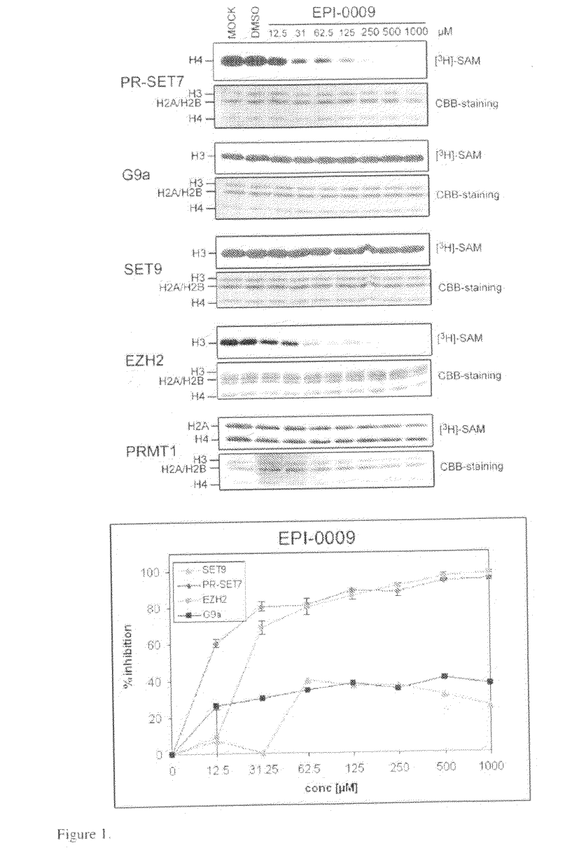

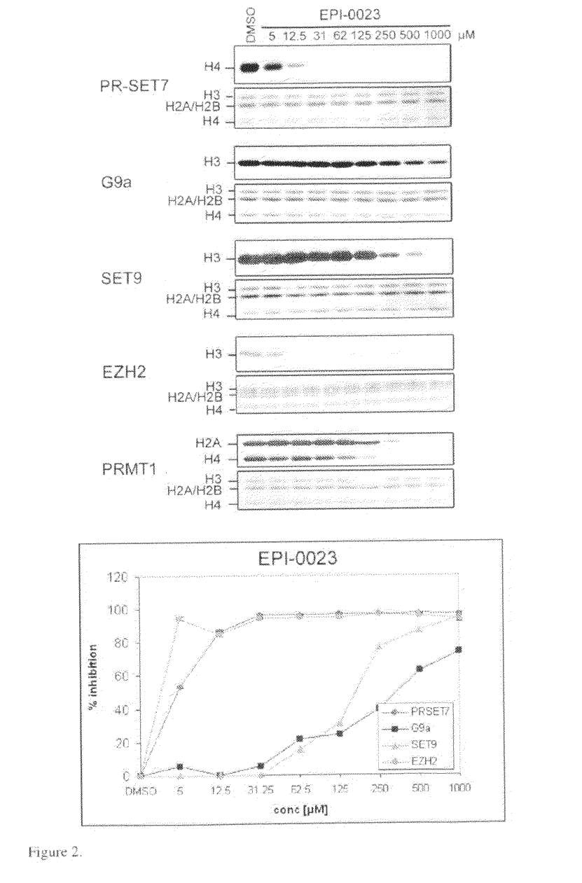

TABLE 1

| Compound | PRSET-7 | G9a | SET7/9 | EZH2 |
|---|---|---|---|---|
| Thymolphthalein | 9.0 | >1450 | >1450 | 25/400 |
| (FIG. 1) | | | | |
| H-acid | 3.8 | >500 | >1380 | 3/>1000 |
| (FIG. 2) | | | | |

The compounds were selected as being specific exclusively for the HKMTs PR-SET7 and EZH2.

### Example 2

### Cell Culture Toxicity and Cell Viability Upon Treatment with Compounds

Compounds that show low IC₅₀ values for specific inhibition of single HKMTs were pre-selected for cell culture based toxicity and growth inhibition. HeLa cells were seeded in a concentration of 1×105 cell/ml in Dulbecco's modified Eagle medium supplemented with 10% Bovine serum, 2 mM glutamine, and penicillin-streptomycin solution. After 24 hours, the medium was supplemented with various compound concentrations. After an additional 48 hours, cells were harvested. Viable cells were stained with Trypan-Blue and counted. Based on the number of living cells treated with a compound in comparison to the cell number of cells treated with DMSO, a toxicity index was calculated for each compound.

### Example 3

### Cell Proliferation Tests and Analysis of Global Changes of Histone Lysine Methylation Patterns Upon Treatment of Cell Culture Cells with Compounds

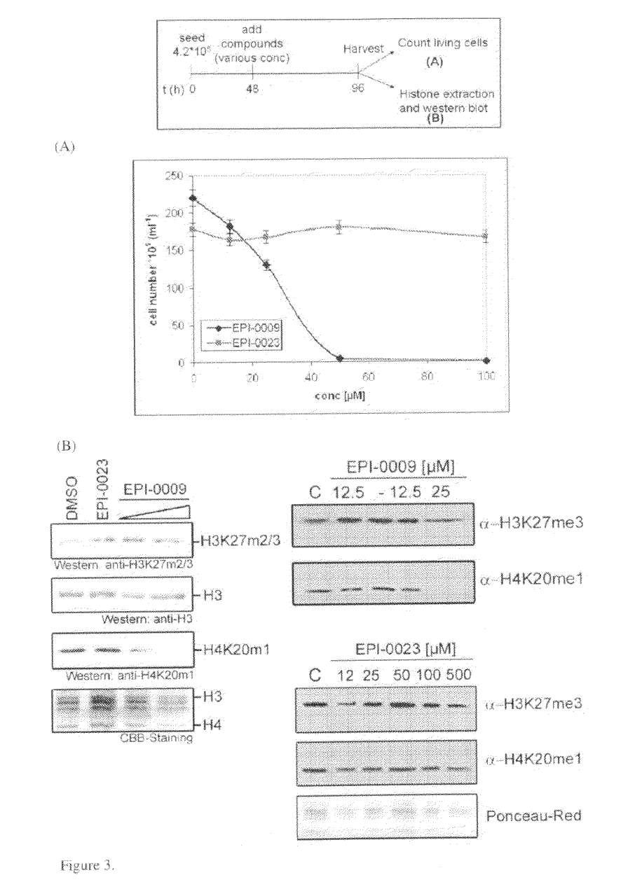

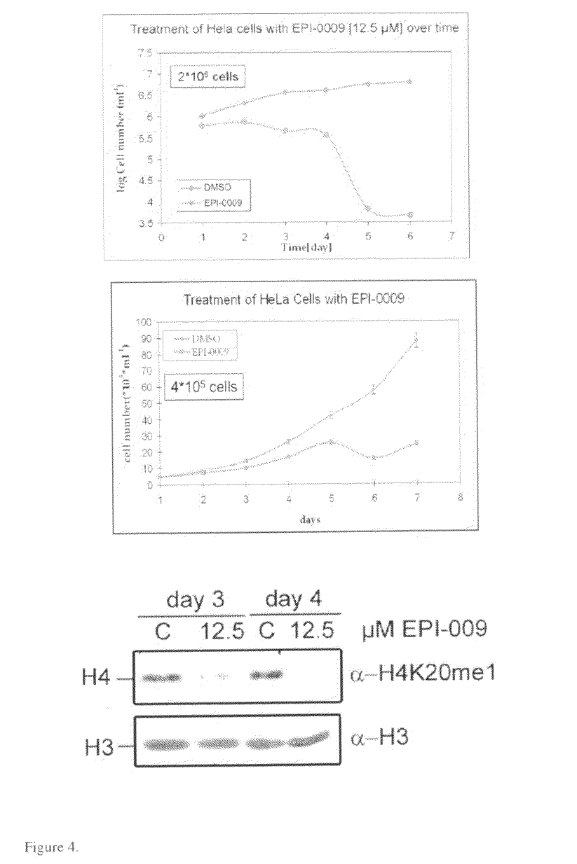

Compounds with a low toxicity index were used for cell viability tests (FIG. 3). The cells harvested from this cell viability test were analyzed using two techniques. In the first technique, living cells were counted as described above and growth curves plotted for cells treated with DMSO or a given compound. (FIG. 4). In the second technique, histones were isolated from cells upon treatment of compounds, and their effect on global histone lysine methylation patterns was analyzed. Harvested cells were subjected to an acid extraction procedure for histones. Cells were washed once with 1 ml PBS and frozen 3× in liquid nitrogen and thawed at 37° C. Pellet was re-suspended in 500 μl of 0.5 M HCl, incubated on ice for 30 minutes and spun at 20,000×g for 10 minutes at 4° C. The supernatant (containing the majority of core histones) was neutralized using 4 M KOH and the addition of 20 μl of 1 M Tris-HCl (pH8.0). Histones were resolved by SDS-PAGE, transferred to nitrocellulose, and analyzed by immunoblotting using histone-modification-state specific antibodies. Based upon these experiments, compounds with low toxicity and compounds exhibiting global inhibition of single histone lysine methylation marks in living cells were selected.

Thymolphthalein showed a reproducible reduction of global histone H4 lysine 20 monomethylation (H4K20me1). Other histone methylation marks (e.g. H3K27me3) were not affected. Because PR-SET7 is the sole enzyme responsible for all H4K20me1 in the cell, the results suggest that thymolphthalein is specifically inhibiting this enzyme.

### Example 4

### Preparation Schemes

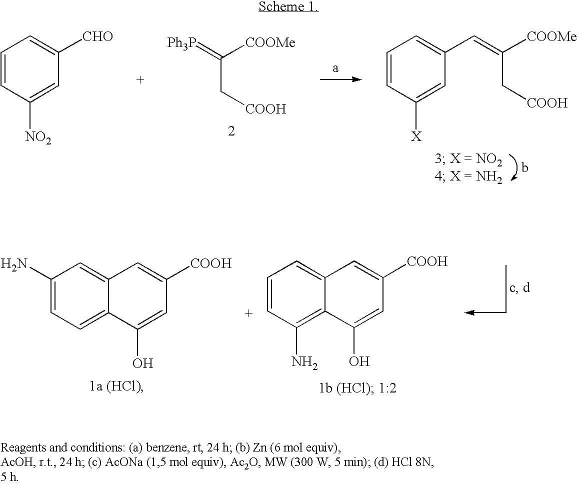

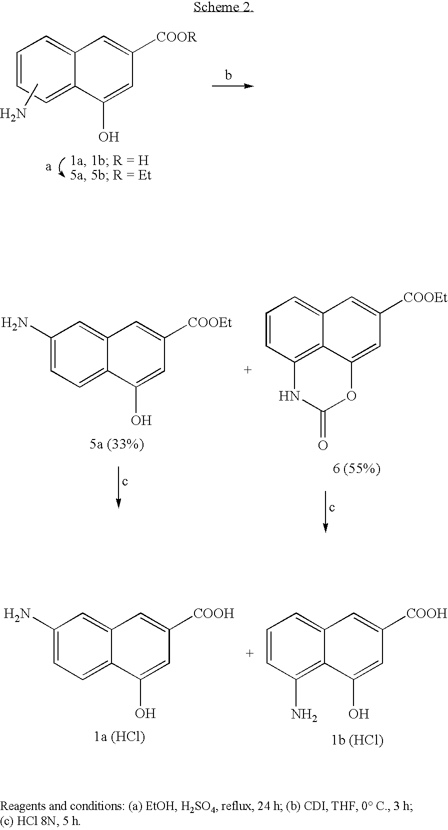

Preparation of (E)-3-(methoxycarbonyl)-4-(3-nitrophenyl)but-3-enoic acid (3). To a suspension of 2 (13.0 g, 35.2 mmol) in dry benzene (150 mL) 3-nitrobenzaldehyde (5.74 g, 38.0 mmol) was added and the resulting mixture was stirred at room temperature for 48 h and then extracted with saturated NaHCO₃solution (3×70 mL). The aqueous phase was washed with ethyl ether, acidified with concentrated HCl and extracted with ethyl acetate (3×60 mL). The combined organic phases were washed with brine and dried. Evaporation of the solvent yielded 8.60 g (92%) of 3 as a white solid which recrystallized from toluene. Mp 160-161° C. (lit. 122-123° C. from benzene). Anal. Calcd for C₁₂H₁₁NO₆: C, 54.34: H, 4.18; N, 5.28. Found: C, 54.44; H, 4.19; N, 5.29. ¹H NMR (300 MHz, CDCl₃) δ 8.30-8.10 (m, 2H), 7.92 (s, 1H), 7.78-7.55 (m, 2H), 3.87 (s, 3H), 3.48 (s, 2H). MS (EI, 70 ev) m/z: 265. ¹H-NMR experiments confirmed the E assignment for compound 3, as the chemical shift of the vinylic hydrogen atom (δ=7.92 ppm) is consistent with a cis position respect to the methoxycarbonyl substituent.

Preparation of (E)-3-(methoxycarbonyl)-4-(3-aminophenyl)but-3-enoic acid (4). To a solution of 3 (2.00 g, 7.54 mmol) in glacial acetic acid (100 mL) Zn dust (3.88 g, 60.3 mmol) was added portion-wise while keeping the temperature below 20° C. with an ice bath. The resulting mixture was vigorously stirred for 24 h. The solids were filtered off and washed with methanol and the combined filtrates were concentrated and the residue was redissolved in ethanol. The white precipitate formed was filtered off and the solvent was evaporated. The TLC pure crude residue was used immediately for subsequent reaction. ¹H NMR (300 MHz, CDCl₃) 7.84 (s, 1H), d 7.22-7.12 (m, 1H), 6.78-6.65 (m, 3H), 5.73 (bra, 3H), 3.84 (s, 3H), 3.58 (s, 2H). MS (EI, 70 ev) m/z: 235.

Preparation of 5-amino- and 7-amino-4-hydroxy-2-naphthoic acids (1a and 1b). A round-bottom flask containing a magnetic stirring bar and fitted with a reflux condenser was charged with a mixture of 4, Ac₂O (35 mL) and NaOAc (1.5 equiv). The flask was placed in a CEM Discover Focused Microwave Synthesis and subjected to MW irradiation (power 300W) for 5 min keeping temperature below 120° C. (air cooling). The crude reaction mixture was evaporated and treated at reflux with HCl 8 N for 5 h. After cooling, the precipitate was collected yielding a pale yellow solid proved to be a 1:2 mixture (NMR determination) of the isomeric amino hydroxyl naphthoic acids hydrochlorides 1a and 1b (70% starting from 3).

Compound 1a (hydrochloride): ¹H-NMR (300 MHz, DMSO-d₆.) δ 10.00 (s, 1H), 7.83 (d, J= 8.8 Hz, 1H), 7.58 (d, J= 1.2 Hz, 1H), 6.98-6.82 (m, 3H), 6.00 (brs, 2H). Mp> 250° C. (dec). Anal. Calcd for CnH9NO₃: C, 65.02; H, 4.46; N, 6.89. Found: C, 64.82; H, 4.45; N, 6.87. MS (+ESI) m/z: 204; Compound 1b (hydrochloride): ¹H-NMR (300 MHz, DMSO-d₆.) δ 10.15 (brs, 1H), 7.90 (d, J= 1.2 Hz, 1H), 7.57 (dd, J= 7.8 Hz, J= 7.8 Hz, 1H), 7.58-6.92 (m, 3H), 5.80 (brs, 2H). Mp> 250° C. (dec). Anal. Calcd for C₁₁H₉NO₃: C. 65.02; H, 4.46; N, 6.89. Found: C. 64.92; H, 4.45; N, 6.88. MS (+ESI) m/z: 204.

Preparation of ethyl 7-amino-4-hydroxynaphthalene-2-carboxylate (5a) and ethyl 2,3-dihydro-2-oxonaphtho[1,8-de][1,3]oxazine-8-carboxylate (6). Triethylamine (0.626 ml, 4.50 mmol) was added to a solution of the esters 5a and 5b (1.30 g, 5.62 mmol) in THF (100 mL) stirred at 0° C. A solution of carbonyldiimidazole (0.730 g, 4.50 mmol) in THF (30 mL) was then added dropwise and the mixture was kept stirring at 0° C. for 3 h. After quenching with water, the mixture was evaporated and the resulting oil was taken up with HCl 1N (50 mL) and extracted with chloroform (3×20 mL). The combined organic layers were washed with HCl 1N (2×10 mL) and with brine, then dried and evaporated to furnish compound 6 (0.795 g, 55%) as a white solid: mp>250° C. (dec); ¹H NMR (300 MHz, CDCl₃) δ 8.95 (brs, 1H), 8.25 (d, J= 1.2 Hz, 1H), 7.56 (d, J= 1.2 Hz, 1H), 7.52 (d, J= 8.4 Hz, 1H), 7.45 (dd, J= 8.4 and 7.2 Hz, 1H), 6.76 (d, 7=7.2 Hz, 1H), 4.44 (q, 7= 7.3 Hz, 2H), 1.34 (t, J= 7.3 Hz, 3H). Anal. Calcd for C₁₄H₁₁NO₄: C, 65.37; H, 4.31; N, 5.44. Found: C, 65.24; H, 4.32; N, 5.43. MS (EI, 70 ev) m/z: 257. The combined aqueous phases were basified with Na₂CO₃ and extracted with ethyl acetate (3×30 mL). The combined organic layers were dried and evaporated to recover unreacted compound 5a (0,430 g, 33%) which was used immediately for subsequent reaction. ¹H NMR (300 MHz, CDCl₃) δ 8.03 (d, J= 8.0 Hz, 1H), 7.92 (d, J= 1.2 Hz, 1H), 7.18 (d, J= 1.2 Hz, 1H), 7.02-6.96 (m, 2H), 5.50 (brs, 2H), 4.40 (q, J= 7.3 Hz, 2H), 1.41 (t, J= 7.3 Hz, 3H). MS (EI, 70 ev) m/z: 231. 5b: ¹H NMR (300 MHz, CDCl₃) δ 8.02 (d, J= 1.2 Hz, 1H), 7.46 (dd, J=8.0 and 1.2 Hz, 1H), 7.37 (d, J=1.2 Hz, 1H), 7.29 (dd, J= 8.0 and 7.8 Hz, 1H), 6.91 (dd, J=7.8 and 1.2 Hz, 1H), 5.50 (brs, 2H), 4.38 (q, J=7.3 Hz, 2H), 1.39 (t, J=7.3 Hz, 3H). MS (EI, 70 ev) m/z: 231.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

1. A pharmaceutical composition comprising:
(a) a compound of Formula I: 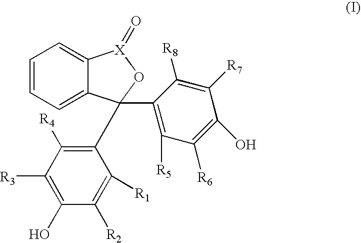
wherein
R₁-R₈ are independently selected from the group consisting of H, (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂Rₐ, SOₘRₐ, S(O)ₘNRₐR_{b}, P(═O)(ORₐ)(Rₐ), and Het, wherein (C₁₋₇)alkyl or (C₃₋₁₂)cycloalkyl are each independently optionally substituted with from 1 to 5 aryl, Het, ORₐ, halo, NO₂, NRₐR_{b}> cyano, CONRₐR_{b}, CO₂Rₐ, SOₘRₐ, S(O)ₘNRₐR_{b}, or P(═O)(ORₐ)(Rₐ);
X is C or S;
Rₐ and R_{b} are each independently H, (C₁₋₇)alkyl, (C₃₋₁₂)cycloalkyl, (C₂₋₇)alkanoyl, (C₂₋₇)alkanoyloxy, or aryl, or Rₐ and R_{b} together with a nitrogen to which they are attached form a Het;
m is 0, 1, or 2;
n is 0, 1, 2, 3, or 4;
or a derivative of said compound selected from the group consisting of N-oxide derivatives, prodrug derivatives, protected derivatives, isomers, and mixtures of isomers of said compound; or a pharmaceutically acceptable salt or solvate of said compound or said derivative; and
(b) a pharmaceutically acceptable carrier.

2. The composition of claim 1, wherein R₁ and R₈ are methyl; R₂, R₄, R₅, and R₇ are hydrogen; and R₃ and are (CH₃)₂CH.

3. A pharmaceutical composition comprising:
(a) a compound of Formula II: 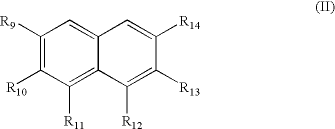
wherein
R₉-R₁₄ are independently selected from the group consisting of H, (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂Rₐ, SOₘRₐ, S(O)ₘNRₐR_{b}, P(═O)(ORₐ)(Rₐ), —N═N-aryl, NHC(═O)Rₐ, Het, and R₁₁ and R₁₂ together are —OC(═O)—NH—, wherein (C₁₋₇)alkyl or (C₃₋₁₂)cycloalkyl are each independently optionally substituted with from 1 to 5 aryl, Het, ORₐ, halo, NO₂, NRₐR_{b}, cyano, CONRₐR_{b}, CO₂Rₐ, SOₘRₐ, S(O)ₘNRₐR_{b}, or P(═O)(ORₐ)(Rₐ);
Rₐ and R_{b} are each independently H, (C₁₋₇)alkyl, (C₃₋₁₂)cycloalkyl, (C₂₋₇)alkanoyl, (C₂₋₇)alkanoyloxy, aryl, or Na;
m is 0, 1, 2, or 3;
n is 0, 1, 2, 3, or 4; or
a derivative of said compound selected from the group consisting of N-oxide derivatives, prodrug derivatives, protected derivatives, isomers, and mixtures of isomers of said compound; or a pharmaceutically acceptable salt or solvate of said compound or said derivative; and
(b) a pharmaceutically acceptable carrier.

4. The composition of claim 3, wherein R₉ is H and R₁₄ is selected from the group consisting of CO₂Rₐ and SOₘRₐ.

5. The composition of claim 3, wherein R₉ is selected from the group consisting of CO₂Rₐ and SOₘRₐ and R₁₄ is H.

6. The composition of claim 3, wherein R₉ and R₁₄ are independently selected from the group consisting of CO₂Rₐ and SOₘRₐ.

7. The composition of claim 3, wherein R₁₀ is H and R₁₃ is —N═N-aryl.

8. The composition of claim 3, wherein R₁₁ and R₁₂ are independently selected from the group consisting of H, OH, SH, NH₂, CO₂Alk, NHC(═O)Alk, and —N═N-aryl.

9. The composition of claim 3, wherein R₁₁ and R₁₂ together are —OC(═O)—NH—.

10. The composition of claim 3, wherein the compound of formula II is:

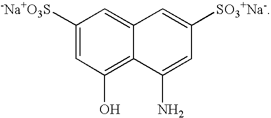

11. The composition of claim 3, wherein the compound of formula II is:

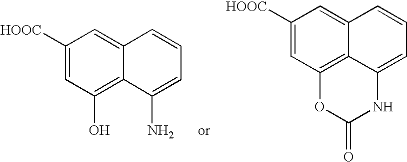

12. A method for treating a proliferative disorder mediated by a methyl transferase comprising administering an anti-proliferative effective amount of the composition of claim 1 to a patient in need thereof.

13. The method of claim 12, wherein said disorder is selected from the group consisting of prostate cancer and breast cancer.

14. The method of claim 12, wherein said methyl-transferase is selected from the group consisting of EZH2 and PRSET7.

15. A compound of formula III: 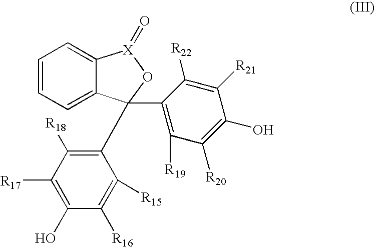
wherein
R₁₅-R₂₂ are independently selected from the group consisting of H, (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂Rₐ, SOₘRₐ, S(O)ₘNRₐR_{b}, P(═O)(ORₐ)(Rₐ), and Het, wherein (C₁₋₇)alkyl or (C₃₋₂)cycloalkyl are each independently optionally substituted with from 1 to 5 aryl, Het, ORₐ, halo, NO₂, NRₐR_{b}, cyano, CONRₐR_{b}, CO₂Rₐ, SOₘRₐ, S(O)ₘNRₐR_{b}, or P(═O)(ORₐ)(Rₐ);
X is C or S;
Rₐ and R_{b} are each independently H, (C₁₋₇)alkyl, (C₃₋₁₂)cycloalkyl, (C₂₋₇)alkanoyl, (C₂₋₇)alkanoyloxy, or aryl, or Rₐ and R_{b} together with a nitrogen to which they are attached form a Het;
m is 0, 1, or 2;
n is 0, 1, 2, 3, or 4;
or a derivative of said compound selected from the group consisting of N-oxide derivatives, prodrug derivatives, protected derivatives, isomers, and mixtures of isomers of said compound; or a pharmaceutically acceptable salt or solvate of said compound or said derivative;
provided that at least one of R₁₅, R₁₈, R₁₉, and R₂₂ is halo.

16. A compound of formula IV: 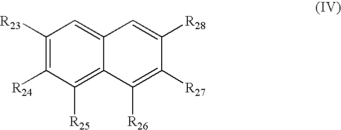
wherein
R₂₃-R₂₈ are independently selected from the group consisting of H, (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂Rₐ, SOₘRₐ, S(O)ₘNRₐR_{b}, P(═O)(ORₐ)(Rₐ), —N═N-aryl, NHC(═O)R_{a, Het, and R}₁₁ and R₁₂ together are —OC(═O)—NH—, wherein (C₁₋₇)alkyl or (C₃₋₁₂)cycloalkyl are each independently optionally substituted with from 1 to 5 aryl, Het, ORₐ, halo, NO₂, NRₐR_{b}, cyano, CONRₐR_{b}, CO₂Rₐ, SOₘRₐ, S(O)ₘ—NRₐR_{b}, or P(═O)(ORₐ)(Rₐ);
Rₐ and R_{b} are each independently H, (C₁₋₇)alkyl, (C₃₋₁₂)cycloalkyl, (C₂₋₇)alkanoyl, (C₂₋₇)alkanoyloxy, aryl, or Na;
m is 0, 1, 2, or 3;
n is 0, 1, 2, 3, or 4; or
a derivative of said compound selected from the group consisting of N-oxide derivatives, prodrug derivatives, protected derivatives, isomers, and mixtures of isomers of said compound; or a pharmaceutically acceptable salt or solvate of said compound or said derivative;
provided that when: (a) at least one of R₂₄ and R₂₇ is H; (b) at least one of R₂₃ and R₂₈ is H, CO₂H, OC(═O)(C₁₋₇)alkyl, or OC(═O)Na, or both R₂₃ and R₂₈ are SO₃Rₐ, and (b) R₂₅ is H, OH, or OC(═O)(C₁₋₇)alkyl and R₂₆ is H, OH, OC(═O)(C₁₋₇)alkyl, or N═N—Ar, wherein Ar is: 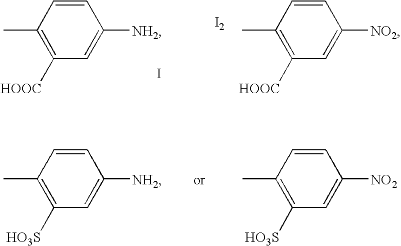
or (c) R₂₅ is NH₂ and Y is H,
then R₂₃ and R₂₈ are independently selected from the group consisting of (C₁₋₇)alkyl, (C₂₋₆)alkenyl, (C₂₋₆)alkynyl, (C₂₋₇)alkanoyl, (C₂₋₇)alkanoyloxy, (C₃₋₁₂)cycloalkyl, (C₁₋₇)acyl, aryl, halo, ORₐ, trifluoromethoxy, trifluoromethyl, NO₂, NRₐR_{b}, cyano, CONRₐR_{b}, CO₂R^{c}, SOₚR^{c}, S(O)ₘNRₐR_{b}, P(═O)(ORₐ)(Rₐ), —N═N-aryl, NHC(═O)Rₐ, Het, and R₂₅ and R₂₆ together are —OC(═O)—NH—,
wherein
(C₁₋₇)alkyl or (C₃₋₁₂)cycloalkyl are each independently optionally substituted with from 1 to 5 aryl, Het, ORₐ, halo, NO₂, NRₐR_{b}, cyano, CONRₐR_{b}, CO₂Rₐ, SOₘRₐ, S(O)ₘNRₐR_{b}, or P(═O)(ORₐ)(Rₐ),
R_{c} is (C₃₋₁₂)cycloalkyl, (C₂₋₇)alkanoyl, (C₂₋₇)alkanoyloxy, or aryl, and
p is 0, 1, or 2.

17. A method for treating a proliferative disorder mediated by a methyl transferase comprising administering an anti-proliferative effective amount of the composition of claim 3 to a patient in need thereof.

18. The method of claim 17, wherein said disorder is selected from the group consisting of prostate cancer and breast cancer.

19. The method of claim 17, wherein said methyl transferase is selected from the group consisting of EZH2 and PRSET7.
